(19) United States
(12) Patent Application Publication
(10) Pub. No.: US 2019/0381015 A1
(43) Pub. Date: Dec. 19, 2019

(54) PHARMACEUTICAL USE OF BERBERINE

(71) Applicant: Shaoliang CHEN, Jiangsu (CN)

(72) Inventor: Shaoliang CHEN, Jiangsu (CN)

(21) Appl. No.: 16/471,625

(22) PCT Filed: Feb. 15, 2017
(86) PCT No.: PCT/CN2017/073620

§ 371 Date: Jun. 20, 2019

(30) Foreign Application Priority Data
Dec. 21, 2016 (CN) 201611189657.2

(51) Int. Cl. A61K 31/4375

(52) U.S. Cl. CPC A61K 31/4375

(57) ABSTRACT

An application of berberine in preparation of drugs for prevention and treatment of pulmonary hypertension, cardiac failure caused by pulmonary hypertension and in-stent restenosis and antithrombotic drugs. Berberine can inhibit stimulation of noradrenaline to pulmonary arterial smooth muscle cells, improve right ventricular function, reduce mean pulmonary arterial pressure, and prevent and treat in-stent restenosis and stent thrombosis.

### BACKGROUND OF THE INVENTION

### Field of the Invention

The present invention relates to chemical drugs, and in particular, relates to pharmaceutical use of berberine.

### Description of Related Art

Pulmonary hypertension: pulmonary hypertension is characterized by progressive increase of pulmonary arterial pressure (mean pulmonary arterial pressure <25 mmHg at normal resting) and pulmonary vascular resistance (<2.5 WU at normal resting), which rapidly leads to right heart failure and early death. The pulmonary artery is a venous blood vessel in which flowing blood has a low oxygen-carrying capacity. Pathological remodeling occurs in the pulmonary artery during pulmonary hypertension, and appears as extreme smooth muscle proliferation, endothelial cellular dysfunction, in-situ thrombosis, and plexiform changes in the media of the distal small pulmonary artery, which significantly increases the pulmonary arterial pressure and pulmonary vascular resistance, thereby causing extreme increase of right ventricular afterload, right ventricular decompensation and right heart failure. Therefore, the target of treating pulmonary hypertension is focused on how to alleviate the pathological remodeling of the pulmonary artery and improve the right heart failure. At present, targeted drugs for treating the pulmonary hypertension mainly include prostacyclins, endothelin receptor antagonists and phosphodiesterase type-5 inhibitors. The above-mentioned targeted drugs have long been monopolized by large pharmaceutical companies in Europe and America. However, the efficacy of these targeted drugs is very limited. For example, after 3-6 months of treatment, the distance in the 6-minute walk test can only increase by 20 to 30 meters, and the mean pulmonary arterial pressure is reduced by 2 to 3 mmHg. Therefore, ultimately, patients can only choose lung transplant surgery for treatment. However, due to very limited donors, most patients die while waiting. To this end, pulmonary hypertension is also known as “a cancer in cardiovascular diseases.” Therefore, it is extremely urgent to research and develop anti-pulmonary hypertension drugs.

In-stent restenosis: atherosclerosis is the leading cause of death, and coronary atherosclerosis (coronary heart disease) is the most common atherosclerotic disease. Percutaneous intracoronary stent implantation is the most effective means of treating coronary heart disease. However, the implanted stents often undergo restenosis and thrombosis, resulting in acute myocardial infarction and the need for revascularization. At present, the anti-platelet drugs in clinical use have reduced the occurrence of in-stent thrombosis, but increased the occurrence of cerebral hemorrhage and massive alimentary tract bleeding. Therefore, to find drugs inhibiting in-stent cell proliferation and platelet aggregation has been the focus of research.

Myocardial infarction: myocardial infarction is a myocardial necrosis caused by a sudden occlusion of the coronary artery. Ventricular systolic function is significantly reduced after myocardial necrosis, leading to cardiac failure, cardiogenic shock, and malignant ventricular arrhythmia, which eventually leads to sudden death. Prevention and treatment of myocardial infarction is a difficult point and focus of clinical treatment. Although drugs including aspirin have the effect of reducing occurrence of myocardial infarction, bleeding complications have always been the main problem restricting the use of such drugs. Therefore, the research and development of new drugs for prevention and treatment of myocardial infarction has always been the core of research.

Ischemic cardiomyopathy and cardiac failure: the coronary heart disease is the leading killer of human beings, and deaths caused by the coronary heart disease rank second in all causes of death. After the coronary heart disease leads to myocardial ischemia, myocardial fibrosis is aggravated, and the number of cardiomyocytes to effectively work is reduced, leading to ischemic cardiomyopathy and cardiac failure, dyspnea and early death. Prevention and treatment of ischemic cardiomyopathy and consequently ventricular remodeling is always the focus of research. β-blockers, calcium ion antagonists, angiotensin converting enzyme inhibitors, and angiotensin receptor antagonists used at present have various side effects, and thus have weak effect of preventing ischemic cardiomyopathy. Therefore, it is very important to research and develop new drugs for prevention and treatment of ischemic cardiomyopathy and cardiac failure.

Berberine: berberine is a common isoquinoline alkaloid with a molecular formula of C₂₀H₁₈NO₄, and exists in many plants of four families and ten genera such as berberidaceae. In 1826, M.-E. Chevallier and G. Pelletan first found berberine from the bark of Xanthoxylon clava. Berberine is a quaternary ammonium alkaloid. Yellow acicular crystals can be precipitated from diethyl ether; it has a melting point of 85-86° C., and is soluble in water and insoluble in benzene, diethyl ether, and chloroform. The solubility of berberine salts in water is relatively low, for example, 1:500 for hydrochloride and 1:30 for sulfate. Berberine has antibacterial activity against hemolytic streptococcus, Staphylococcus aureus, Neisseria gonorrhoeae, Freund's, Shigella dysenteriae, etc., and enhances phagocytosis of leucocytes, and also has inhibiting effects on Mycobacterium tuberculosis and Yersinia pestis as well as amoeba in rats. Berberine has an anti-curare effect in animals and has a peripheral antihypertensive and antipyretic effect. The hydrochloride of berberine (commonly known as berberine hydrochloride) has been widely used in the treatment of gastroenteritis, bacterial dysentery, etc., and has certain curative effects on tuberculosis, scarlatina, acute tonsillitis, and respiratory infections. Berberine also has the effect of lowering blood lipids and regulating blood sugar.

### SUMMARY OF THE INVENTION

### Technical Problem

A first objective of the present invention is to provide pharmaceutical use of berberine for pulmonary hypertension, a second objective of the present invention is to provide another pharmaceutical use of berberine for in-stent thrombosis, a third objective of the present invention is to provide another pharmaceutical use of berberine for myocardial infarction, and a fourth objective of the present invention is directed to ischemic cardiomyopathy and consequently cardiac failure.

### Technical Solution

An application of berberine in preparation of drugs for prevention and treatment of pulmonary hypertension and related disorders thereof. The disorder related to pulmonary hypertension is cardiac failure after myocardial infarction caused by pulmonary hypertension. The cardiac failure comprises right cardiac failure and left cardiac failure.

A drug for prevention and treatment of pulmonary hypertension and related disorders thereof, wherein an effective ingredient comprises berberine.

A drug for prevention and treatment of cardiac failure caused by pulmonary hypertension, wherein an effective ingredient comprises berberine.

An application of berberine in preparation of drugs for prevention and treatment of in-stent restenosis.

The in-stent restenosis is restenosis caused by thrombosis.

A drug for prevention and treatment of in-stent restenosis, wherein an effective ingredient comprises berberine.

A drug for prevention and treatment of thrombosis, wherein an effective ingredient comprises berberine.

### Advantageous Effect

Berberine has a significant effect of inhibiting stimulation of noradrenaline to pulmonary arterial smooth muscle cells, can effectively improve right ventricular function, and can significantly reduce the mean pulmonary arterial pressure, and also has the effects of prevention and treatment of in-stent restenosis and in-stent thrombosis. Berberine has a unique effect of prevention and treatment of myocardial infarction, and can effectively prevent and treat ischemic cardiomyopathy and consequently cardiac failure.

### BRIEF DESCRIPTION OF THE DRAWINGS

FIG. 1 is a schematic diagram of berberine inhibiting noradrenaline. The pulmonary arterial smooth muscle cells are stimulated with noradrenaline (NE) for 2 hours and then randomly divided into a berberine group (Ctl group, berberine for 4 hours) and a control group. In the control group, cells are collected at various time points (30 minutes-4 hours) after the grouping, and the protein phosphorylation activities of liver kinase B1 (LKB1) and G-protein coupled receptor kinase 2 (Grk2) are measured. GAPDH is an internal reference protein.

FIG. 2 is a mirror view for a contrast experiment of berberine inhibiting pulmonary artery remodeling. Animals with pulmonary hypertension are randomly divided into a normal control group, a berberine group, and a blank group without treatment. Histochemical staining (HE) and vascular endothelial vWF staining, smooth muscle cell staining (SMA). At 14 weeks, the results show that the pathological remodeling of pulmonary vasculature in the berberine group is significantly reduced.

FIG. 3 is a histogram showing that berberine significantly increases the right ventricular systolic displacement amplitude (TAPSE). The right ventricular function in the berberine treatment group is significantly improved.

FIG. 4 is a histogram of contrast experiment data showing that berberine reduces the mean pulmonary arterial pressure. The mean pulmonary arterial pressure in the berberine treatment group is significantly reduced. Every three data columns in the figure are a group, and are a normal group, a blank group, and a berberine group from left to right in sequence.

FIG. 5 is an experimental control diagram of berberine in the prevention and treatment of in-stent restenosis. For a 79-year-old female patient with stenocardia, coronary angiography before stenting shows severe left main trunk stenosis, and the stenosis substantially disappears immediately after stenting. Stenocardia recurs at the 4th month after surgery, and coronary angiography shows severe in-stent restenosis at the opening of anterior descending branch and in the proximal end of circumflex artery, and involves left main trunk. After continuous berberine treatment for 6 months, coronary angiography shows that the in-stent restenosis substantially disappears.

FIG. 6 is an experimental result diagram showing berberine for prevention and treatment of ischemic cardiomyopathy. Fluorescent staining (left) and electron microscopy (right A-C) show that apoptosis of myocardial cells in the berberine treatment group is significantly reduced.

FIG. 7 is a research result diagram showing that berberine significantly improves cardiac failure caused by ischemic cardiomyopathy. Ultrasonic cardiography of one patient with ischemic cardiomyopathy day 22 after myocardial infarction shows severe ventricular dysfunction. After the patient with ischemic cardiomyopathy is continuously administered with berberine for 3 months, the left ventricular ejection fraction is significantly increased, and myocardial systolic function is improved.

### DETAILED DESCRIPTION OF THE INVENTION

The following examples are intended to further illustrate the content of the present invention, and should not be construed as limiting the present invention. Modifications and replacements of methods, steps or conditions of the present invention without departing from the spirit and scope of the present invention fall within the scope of the present invention. The technical means used in the examples are conventional means well known to those skilled in the art unless otherwise specified.

### EXAMPLE 1

Prevention and Treatment of Pulmonary Hypertension and Right Cardiac Failure

Cell Experiment:

Pulmonary arterial smooth muscle cells in normal animals were selected and showed that the most common, highly expressed Grk2 and LKB1 of the right cardiac failure were significantly increased after noradrenaline stimulation for various time periods, while the (CTL) group showed a significant decrease after berberine is added for 30 minutes at the same time. The results show that berberine has a significant effect of inhibiting stimulation of noradrenaline to pulmonary arterial smooth muscle cells (FIG. 1).

Animal Experiment:

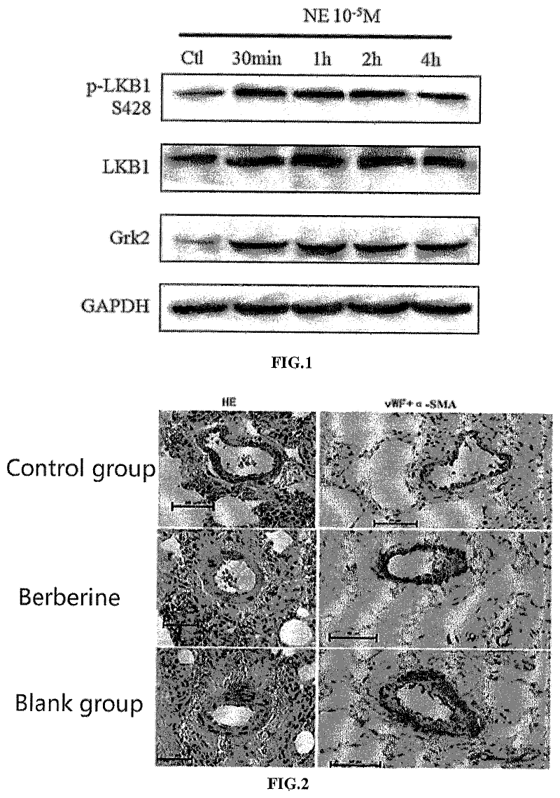

20 beagles were selected, were subjected to pulmonary hemodynamics measurement, and then were randomly divided into a berberine group (intragastric gavage, 0.1 g/d for 14 days) and a control group (intragastric gavage of a berberine coating, without berberine, 14 days in total). At day 14, dehydrogenated monocrotaline (60 mg/kg) was injected into the right atrium, observation was carried out for 8 weeks, and the pulmonary hemodynamics was retested. Results: the mean pulmonary arterial pressure of only one animal in the berberine group reaches 28 mmHg, and the mean pulmonary arterial pressures of the other 9 animals are all <25 mmHg (19±3.0 mmHg). In contrast, the mean pulmonary arterial pressures of 8 animals in the control group are all >26 mmHg (28.4±2.1 mmHg), one animal for 24.6 mmHg, and the other one for 24.8 mmHg. The results show that berberine has the effect of inhibiting dehydrogenated monocrotaline to induce pulmonary hypertension. Subsequently, 8 animals in the control group with the mean pulmonary arterial pressure of >25 mmHg were administered by intragastric gavage with berberine (0.1 g/d for a total of 42 days), and the proliferation degree of pulmonary arterial smooth muscle was significantly reduced (FIG. 2).

Clinical Experiment:

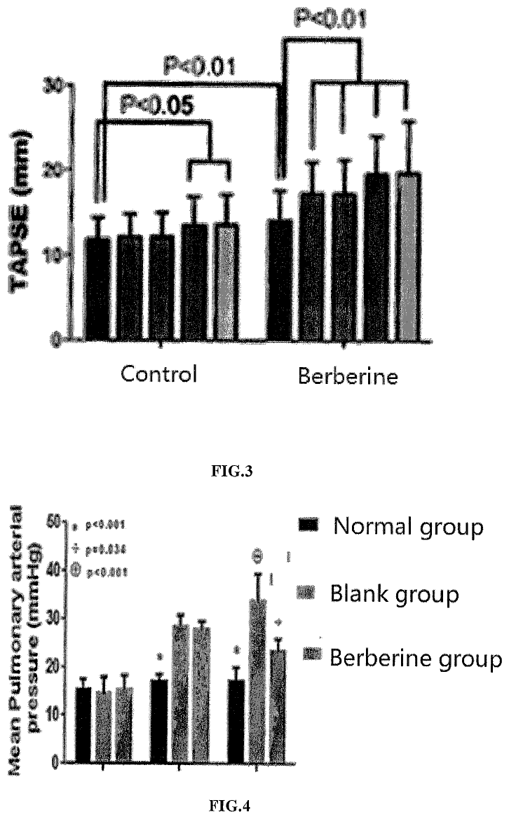

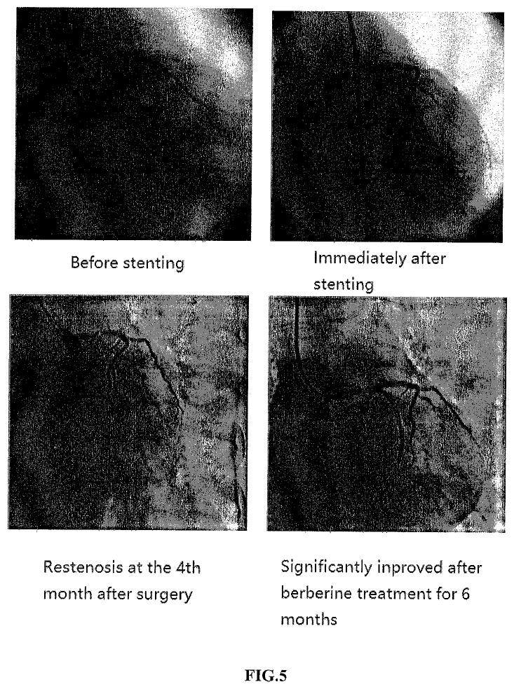

Six patients with idiopathic pulmonary hypertension were divided into two groups (3 in each group): a control group (administered with targeted drugs according to the original doses) and a berberine group (based on the original targeted drugs, additionally administered with berberine at 0.3 g/d for a total of 3 months). After 3 months of treatment, the right ventricular function of the berberine group was significantly improved (FIG. 3), and the mean pulmonary arterial pressure was significantly reduced (FIG. 4).

### EXAMPLE 2

Prevention and Treatment of In-stent Restenosis and In-stent Thrombosis

100 patients with drug-eluting stent implantation were randomly divided into two groups: a control group (without additional berberine) and a berberine group (additionally administered with berberine at 0.3 g/d since the 3th month after surgery for a total of 6 months), and coronary angiography was performed again at the 12th month after surgery. Results: the proportion of in-stent restenosis in the control group is 14% (N=7), and only 2 cases in the berberine group show restenosis (4%, p=0.035) and the degree of stenosis is significantly lower than that in the control group (58±4% vs. 76.3±9%, p=0.029). 7 patients with restenosis in the control group were orally administrated with berberine (0.3 g/d for a total of 6 months) and further followed up for 9 months, and the results show that the stenosis degree of 6 patients is significantly reduced (FIG. 5).

### EXAMPLE 3

Prevention and Treatment of Myocardial Infarction

30 animals in the myocardial infarction model were randomly divided into a control group and a berberine group by intragastric gavage. The results show that berberine (0.3 g, three times a day, three months later) significantly reduces apoptosis of myocardial cells (left panel of FIG. 6), and electron microscopy confirms a decrease in apoptotic bodies (A-C in right panel of FIG. 6). The cardiomyocytes in the berberine treatment group are arranged intactly.

### EXAMPLE 4

Prevention and Treatment of Cardiac Failure Caused by Ischemic Cardiomyopathy

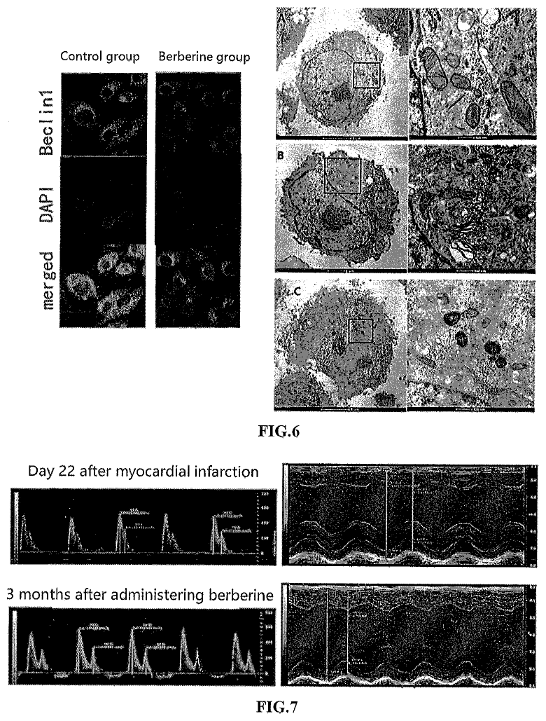

40 patients with acute myocardial infarction were randomly divided into a control group and a berberine group (0.3 g, three times a day, three months later). The mean left ventricular ejection fraction of the control group is 41%, while the mean left ventricular ejection fraction of the berberine group is increased to 53% (p=0.037). FIG. 7 shows that in one patient with acute anterior myocardial infarction day 22 after myocardial infarction, the left ventricle is significantly enlarged, the ejection fraction is decreased, and the myocardial contraction is flat (upward); after berberine treatment for 3 months, the cardiac cavity is reduced, the ejection fraction is significant increased, and the myocardial contractility is significantly enhanced (downward).

1. Method for prevention and treatment of pulmonary hypertension and related disorders thereof comprising the step of:
providing berberine to a user for the treatment of pulmonary hypertension.

2. The process according to claim 1, wherein the process comprises the step of applying berberine to treat a disorder related to pulmonary hypertension which is cardiac failure after myocardial infarction caused by pulmonary hypertension.

3. The process as in claim 2, wherein the cardiac failure comprises right cardiac failure and left cardiac failure.

4. A drug for prevention and treatment of pulmonary hypertension and related disorders thereof, wherein an effective ingredient comprises berberine.

5. The drug as in claim 4, wherein the drug is configured for a treatment of cardiac failure caused by pulmonary hypertension, wherein an effective ingredient comprises berberine.

6. The drug as in claim 4 wherein the drug is configured for prevention and treatment of in-stent restenosis.

7. The drug according to claim 6, wherein the in-stent restenosis is restenosis caused by thrombosis.

8. A drug for prevention and treatment of in-stent restenosis, wherein an effective ingredient comprises berberine.

9. The drug as in claim 8, wherein the drug is used to treat thrombosis.
